(19) United States
(12) Patent Application Publication
(10) Pub. No.: US 2025/0069748 A1
(43) Pub. Date: Feb. 27, 2025

(54) MEDICAL INFORMATION DISPLAY CONTROL APPARATUS, MEDICAL INFORMATION CONTROL METHOD, AND X-RAY DIAGNOSIS SYSTEM

(71) Applicant: Canon Medical Systems Corporation, Otawara-shi (JP)

(72) Inventor: Shumpei OHASHI, Otawara (JP)

(73) Assignee: Canon Medical Systems Corporation, Otawara-shi (JP)

(21) Appl. No.: 18/810,605

(22) Filed: Aug. 21, 2024

(30) Foreign Application Priority Data
Aug. 25, 2023 (JP) 2023-137354
Aug. 6, 2024 (JP) 2024-130046

(51) Int. Cl. G16H 50/20; G16H 30/40

(52) U.S. Cl. CPC G16H 50/20; G16H 30/40

(57) ABSTRACT

According to one embodiment, a medical information display control apparatus includes processing circuitry. The processing circuitry is configured to accept a pointer operation signal corresponding to an operation by a user, acquire a plurality of medical information screen data laid out as a display screen, detect a first pointer drawn in one of the plurality of medical information screen data and specify first pointer coordinates indicating a position of the first pointer, calculate second pointer coordinates indicating a position of a second pointer drawn on the display screen based on the pointer operation signal, and output a pointer movement signal based on the pointer operation signal so as to substantially match the first pointer coordinates with the second pointer coordinates.

### CROSS-REFERENCE TO RELATED APPLICATION(S)

This application is based upon and claims the benefit of priority from the prior Japanese Patent Application No. 2023-137354, filed Aug. 25, 2023, and No. 2024-130046, filed Aug. 6, 2024, the entire contents of all of which are incorporated herein by reference.

### FIELD

Embodiments described herein relate generally to a medical information display control apparatus, a medical information control method, and an X-ray diagnosis system.

### BACKGROUND

A catheter room in which the imaging apparatus of an X-ray diagnosis system is accommodated is configured to use a large screen monitor for displaying an integrated screen formed by integrating the screens of a plurality of devices using a compositor. The screens of a plurality of devices, applications, and the like are laid out on the large screen monitor. The large screen monitor is preferably configured to move a mouse pointer across the respective screens in the integrated screen in accordance with a mouse operation by the user.

Accordingly, some compositors include a KVM (Keyboard/Video/Mouse) function. The KVM function is a function of relaying an input from an HID (Human Interface Device) such as a mouse connected to the compositor to a plurality of devices, a large screen monitor, and the like.

In this case, in general, a mouse input signal is a digital signal corresponding to a two-dimensional movement amount. The coordinates of a mouse pointer on a screen in an application in a given device are mainly calculated by the OS (Operating System) of the device as the accumulation of pointer movement amounts obtained by multiplying movement amounts corresponding to mouse input signals by a magnification ratio. The OS decides this magnification ratio in accordance with settings, the acceleration of the mouse, and the like.

Devices and applications that display screens via a compositor can be own company's devices, own company's applications, and other company's devices. In using own company's devices and own company's applications, installing desired software makes it possible to draw a mouse pointer at arbitrary coordinates calculated by a compositor based on coordinates on a large screen monitor.

In contrast, in using other company's devices, since desired software cannot be installed, a mouse pointer cannot be drawn at arbitrary coordinates calculated by a compositor. For example, in using other company's devices, since a magnification ratio applied to a movement amount is unknown, the compositor cannot calculate the coordinates of the mouse pointer.

Accordingly, in moving a mouse pointer from an own company's device screen to an other company's device screen on a large screen monitor, it is necessary to perform a (jump-in) operation of transferring the control of the mouse point to the other company's device and a (jump-out) operation of returning the control of the mouse pointer to the compositor (i.e., onto the large screen). Such operations cause a deterioration in operability with respect to the mouse pointer that moves across a plurality of screens.

### BRIEF DESCRIPTION OF THE DRAWINGS

FIG. 1 is a block diagram showing an example of a compositor and its peripheral arrangement according to the first embodiment.

FIG. 2 is a schematic view for explaining an example of global coordinates and local coordinates according to the first embodiment.

FIG. 3 is a flowchart for explaining an operation according to the first embodiment.

FIG. 4 is a schematic view showing an example of a display screen on which medical information screens are laid out for the explanation of an operation according to the first embodiment.

FIG. 5 is a schematic view showing an example of a display screen on which medical information screens are laid out for the explanation of an operation according to the first embodiment.

FIG. 6 is a flowchart for explaining an example of an operation in step ST8 in FIG. 3.

FIG. 7 is a schematic view showing an example of a display screen on which medical information screens are laid out for the explanation of an operation according to the first embodiment.

FIG. 8 is a schematic view showing an example of a display screen on which medical information screens are laid out for the explanation of an operation according to the first embodiment.

FIG. 9 is a schematic view showing an example of a display screen on which medical information screens are laid out for the explanation of an operation according to the first embodiment.

FIG. 10 is a schematic view showing an example of a medical information screen of an external device and a display screen of a display for the explanation of an operation according to a modification of the first embodiment.

FIG. 11 is a schematic view showing an example of a medical information screen of an external device and a display screen of a display for the explanation of an operation according to a modification of the first embodiment.

FIG. 12 is a schematic view showing an example of a display screen on which medical information screens are laid out for the explanation of an operation according to another modification of the first embodiment.

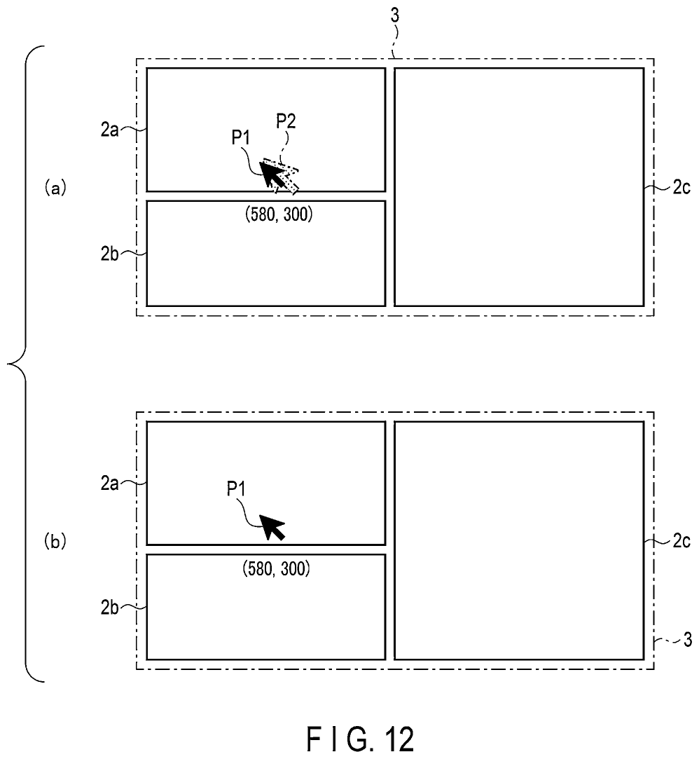

FIG. 13 is a block diagram showing an example of a compositor and its peripheral arrangement according to still another modification of the first embodiment.

FIG. 14 is a block diagram showing an example of an X-ray diagnosis system and its peripheral arrangement according to the second embodiment.

FIG. 15 is a schematic view for explaining boundary neighborhoods according to the third embodiment.

FIG. 16 is a flowchart for explaining an operation according to the third embodiment.

FIG. 17 is a schematic view showing an example of a display screen on which medical information screens are laid out for the explanation of an operation according to the third embodiment.

### DETAILED DESCRIPTION

In general, according to one embodiment, a medical information display control apparatus includes processing circuitry. The processing circuitry is configured to accept a pointer operation signal corresponding to an operation by a user. The processing circuitry is configured to acquire a plurality of medical information screen data laid out as a display screen. The processing circuitry is configured to detect a first pointer drawn in one of the plurality of medical information screen data and specify first pointer coordinates indicating a position of the first pointer. The processing circuitry is configured to calculate second pointer coordinates indicating a position of a second pointer drawn on the display screen based on the pointer operation signal. The processing circuitry is configured to output a pointer movement signal based on the pointer operation signal so as to substantially match the first pointer coordinates with the second pointer coordinates.

Each embodiment will be described below with reference to the accompanying drawings. A compositor will be described below by exemplifying a medical information display control apparatus.

### First Embodiment

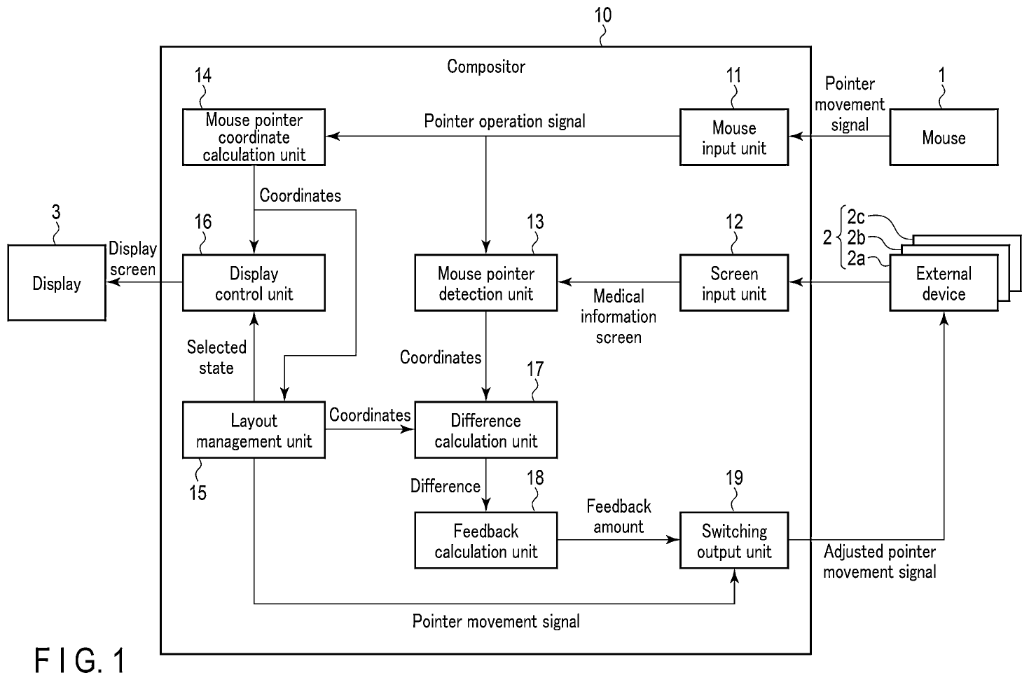

FIG. 1 is a block diagram showing an example of a compositor and its peripheral arrangement according to the first embodiment. Referring to FIG. 1, a mouse 1, a plurality of external devices 2a to 2c, and a display 3 are connected to a compositor 10. The plurality of external devices 2a to 2c will be denoted by the same reference numeral and described as external devices 2 unless they are discriminated from each other.

The mouse 1 is an input device for operating a mouse pointer on a screen and inputs a mouse input signal to the compositor in accordance with an operation by the operator. Note that an input device is not limited to the mouse 1, and any device that can operate a pointer on a screen can be used as an input device, such as a trackball or touch pad, as needed.

Each of the external devices 2a to 2c generates and displays medical information screen data on a screen by causing a processor to execute the installed OS (Operating System) and application and transmits the medical information screen data to the compositor 10. At this time, one of the external devices 2a to 2c generates medical information screen data with a mouse pointer drawn on a medical information screen based on a pointer movement signal input from the compositor 10. Note, however, that such one of the external devices 2a to 2c changes to another one depending on the pointer coordinates. In addition, each of the external devices 2a to 2c calculates the coordinates of the pointer on a medical information screen as the accumulation of pointer movement amounts obtained by multiplying the movement amounts corresponding to pointer movement signals by a magnification ratio and draws a pointer at the position indicated by the coordinates. This magnification ratio is decided by the OS of each of the external devices 2a to 2c based on settings and the acceleration of the mouse. Assume that the external devices 2a to 2c respectively use different magnification ratios in this embodiment. In addition, the term “external device” represents a device arranged outside the compositor 10 and configured to display a medical information screen and hence may be read as “medical device”, “device”, or the like. In other words, the term “external device” is not a term indicating an other company's device. The external device 2 is an example of a medical device.

The display 3 is constituted by a display body that displays a display screen, an internal circuit that supplies a display signal to the display body, and peripheral circuits such as connectors and cables that connect the display to the internal circuit. For example, the display 3 displays a display screen based on the display screen data transmitted from the compositor 10. The display 3 displays a display screen (composite screen) on which medical information screens from the respective external devices 2 are laid out and hence may called a “large screen monitor” or “monitor”.

The compositor 10 includes a mouse input unit 11, a screen input unit 12, a mouse pointer detection unit 13, a mouse pointer coordinate calculation unit 14, a layout management unit 15, a display control unit 16, a difference calculation unit 17, a feedback calculation unit 18, and a switching output unit 19.

The mouse input unit 11 accepts a pointer movement signal corresponding to the operation of the mouse 1 by the user as a pointer operation signal (x/y movement amounts). In this case, x/y movement amounts correspond to an input mouse movement amount in the X direction and an input mouse movement amount in the y direction. The operation of the mouse 1 corresponding to a pointer movement signal includes, for example, at least a moving operation of a moving operation and a pausing operation. The moving operation is an operation of moving the mouse 1. The mouse 1 in motion generates a pointer movement signal corresponding to the x/y movement amounts based on the moving operation and inputs the signal to the mouse input unit 11. The pausing operation is an operation of maintaining the resting state of the mouse 1. The x/y movement amounts of the mouse 1 in the resting state each are zero, and hence no pointer movement signal is generated and input to the mouse input unit 11. This is not exhaustive. In performing a pausing operation, a pointer movement signal may be generated with each of x and y movement amounts being zero, and the mouse input unit 11 may accept the pointer movement signal as a pointer operation signal (0, 0). Alternatively, in performing a pausing operation, since no pointer movement signal is input to the mouse input unit 11, the mouse input unit 11 may not accept any pointer operation signal. In addition, the mouse input unit 11 accepts a click operation signal in accordance with the click operation of the mouse by the user. The mouse input unit 11 is an example of an acceptance unit.

The screen input unit 12 acquires a plurality of medical information screen data laid out as a display screen from the plurality of external devices 2. More specifically, the screen input unit 12 individually acquires a plurality of medical information screen data from the plurality of external devices 2. The screen input unit 12 is an example of an acquisition unit.

The mouse pointer detection unit 13 detects a first mouse pointer (to be referred to as the first pointer hereinafter) drawn on one of a plurality of medical information screen data and specifies first pointer coordinates (local coordinates) indicating the position of the first pointer. For example, the mouse pointer detection unit 13 may detect the first pointer from medical information screen data based on the shape data of the first pointer. In addition, for example, the mouse pointer detection unit 13 may detect the first pointer based on a change in medical information screen data along the time series and a pointer operation signal. In the latter case, the mouse pointer detection unit 13 detects, as the first pointer, an object, of objects moving along the time series within medical information screen data, which has high correlation with a pointer operation signal. The mouse pointer detection unit 13 is an example of a detection unit.

The mouse pointer coordinate calculation unit 14 calculates second pointer coordinates (global coordinates) indicating the position of the second mouse pointer (to be referred to as the second pointer hereinafter) drawn on a display screen based on a pointer operation signal. The mouse pointer coordinate calculation unit 14 is an example of a calculation unit.

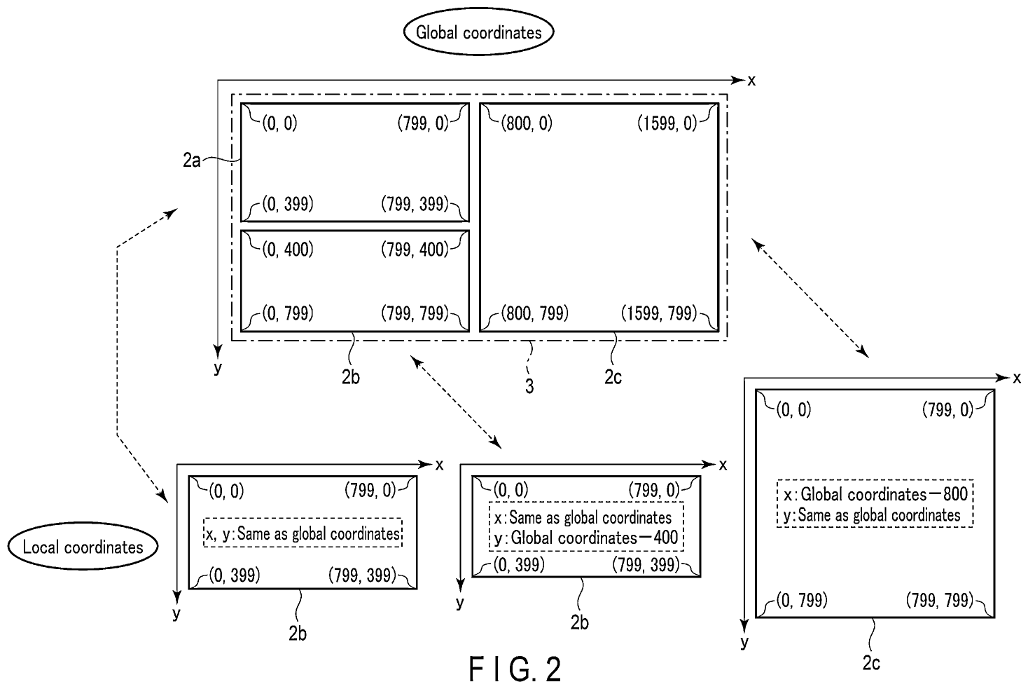

The layout management unit 15 holds layout information indicating the layout of a plurality of medical information screen data. The layout management unit 15 identifies a medical information screen including the second pointer from the second pointer coordinates (global coordinates) calculated by the mouse pointer coordinate calculation unit 14 and held layout information. The layout management unit 15 converts the calculated second pointer coordinates (global coordinates) into local coordinates on the medical information screen including the second pointer. For example, as shown in FIG. 2, global coordinates on a display screen (whole screen) displayed on the display 3 and local coordinates on a medical information screen (window screen) displayed on each of the external devices 2a to 2c can be converted from each other. More specifically, first pointer coordinates are local coordinates indicating a position on medical information screen data. Second pointer coordinates are global coordinates indicating a position on a display screen. The layout management unit 15 associates local coordinates on each of a plurality of medical information screen data with global coordinates on a display screen based on layout information, thereby converting the second pointer coordinates as global coordinates into the local coordinates. Note that the mouse pointer coordinate calculation unit 14 may execute the processing of converting second pointer coordinates (global coordinates) into second pointer coordinates (local coordinates). In any case, the obtained second pointer coordinates (local coordinates) are transmitted to the difference calculation unit 17. In addition, the layout management unit 15 transmits a pointer movement signal corresponding to a pointer operation signal to the switching output unit 19. The layout management unit 15 may control the switching output unit 19, based on layout information, to stop outputting a pointer movement signal to the external device 2 corresponding to medical information screen data having undergone a transition from a state including second pointer coordinates to a state including no second pointer coordinates.

The display control unit 16 causes the display 3 to display a display screen based on a plurality of medical information screen data laid out in accordance with layout information and display screen data including the second pointer drawn in accordance with second pointer coordinates (global coordinates). In this case, since the first pointer is drawn on one of the medical information screen data, the first pointer and the second pointer are drawn on the display screen. However, this is not exhaustive, and the display control unit 16 may end drawing the second pointer when the first pointer coordinates almost match the second pointer coordinates. In addition, the display control unit 16 may draw a second pointer having a size different from that of a first pointer and a shape similar to that of the first pointer. The display control unit 16 may draw a second pointer having a color different from that of a first pointer. That is, a first pointer and a second pointer may have similar shapes with different sizes. In addition, a first pointer and a second pointer may have different colors. Furthermore, a first pointer and a second pointer may have similar shapes with different sizes and colors.

The difference calculation unit 17 calculates the difference between the first pointer coordinates (local coordinates) specified by the mouse pointer detection unit 13 and the second pointer coordinates (local coordinates) converted by the layout management unit 15.

The feedback calculation unit 18 calculates a feedback amount based on the difference so as to almost match the first pointer coordinates with the second pointer coordinates. For example, the feedback calculation unit 18 calculates a feedback amount so as to bring the difference close to zero based on the previous difference and the ratio between the previous input mouse movement amount and the output mouse movement amount. A feedback amount is a movement amount ratio for bringing the difference close to zero.

The switching output unit 19 adjusts the pointer movement signal transmitted by the layout management unit 15 based on the feedback amount calculated by the feedback calculation unit 18 and outputs the adjusted pointer movement signal to one of the external devices 2. For example, the switching output unit 19 adjusts the pointer movement signal so as to almost match the first pointer coordinates with the second pointer coordinates and outputs the adjusted pointer movement signal. In addition, for example, the switching output unit 19 may start to output a pointer movement signal in response to a click operation signal. For example, upon detection of clicking within a layout boundary in the display screen of the display 3, the switching output unit 19 may start to output a pointer movement signal to the external device 2 assigned to the layout. Note that the difference calculation unit 17, the feedback calculation unit 18, and the switching output unit 19 each are an example of an output unit that outputs a pointer movement signal based on an pointer operation signal so as to almost match the first pointer coordinates with the second pointer coordinates. In addition, the difference calculation unit 17, the feedback calculation unit 18, and the switching output unit 19 each are an example of an output unit that outputs a pointer movement signal so as to almost match the first pointer coordinates with the second pointer coordinates as the converted local coordinates. In this case, “almost match” means that the first pointer coordinates match the second pointer coordinates within a reference range. A state in which the first and second pointer coordinates match within the reference range corresponds to, for example, a case where the distance between the first pointer coordinates and the second pointer coordinates is small, as well as zero, so that the first pointer and the second pointer overlap each other at least partly. In addition, the switching output unit 19 is an example of an output unit that starts to output a pointer movement signal in response to a click operation signal.

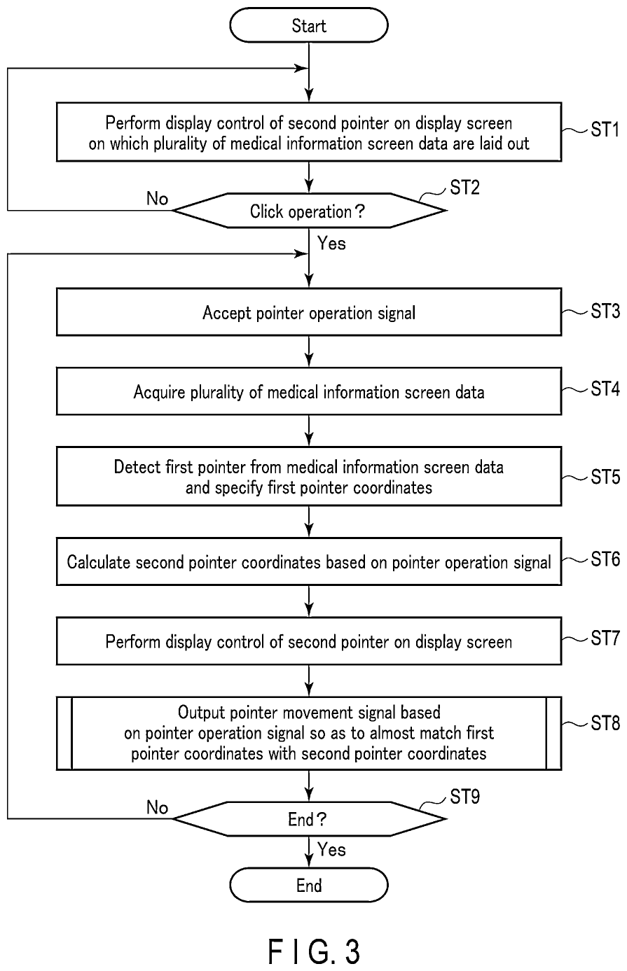

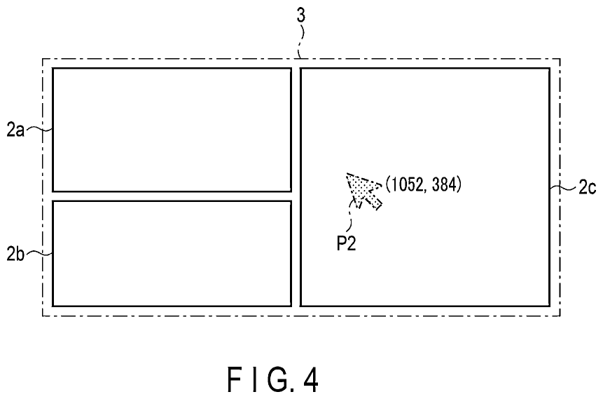

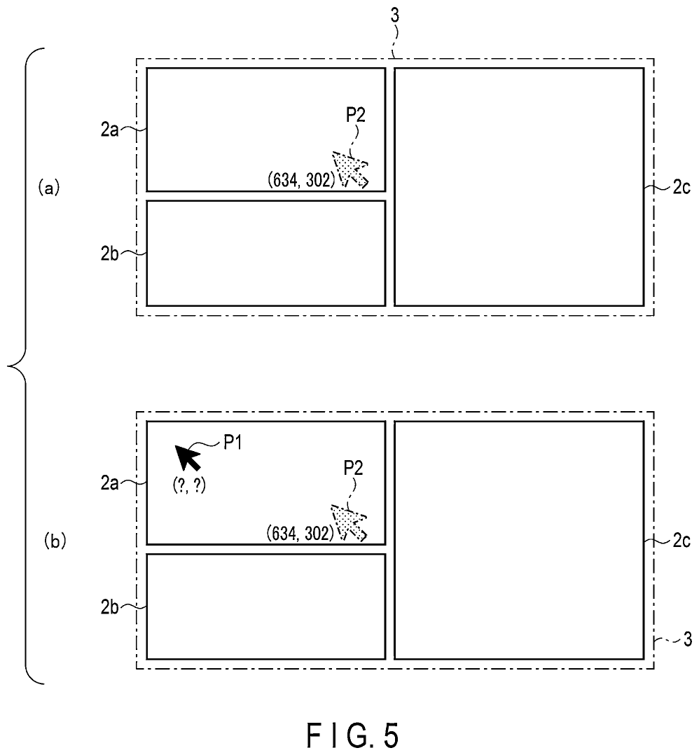

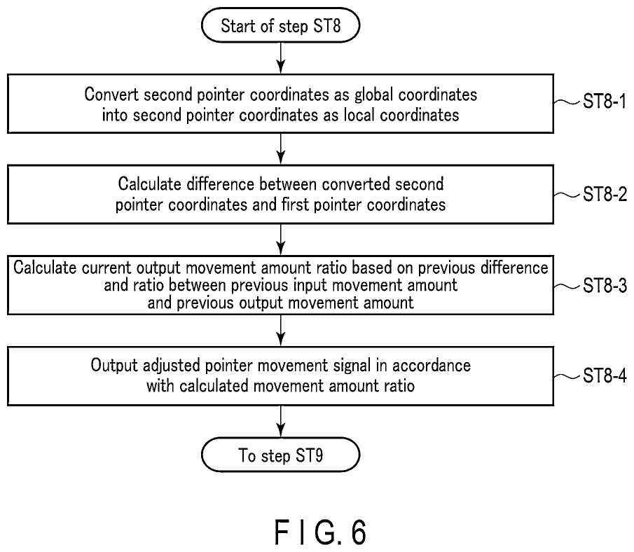

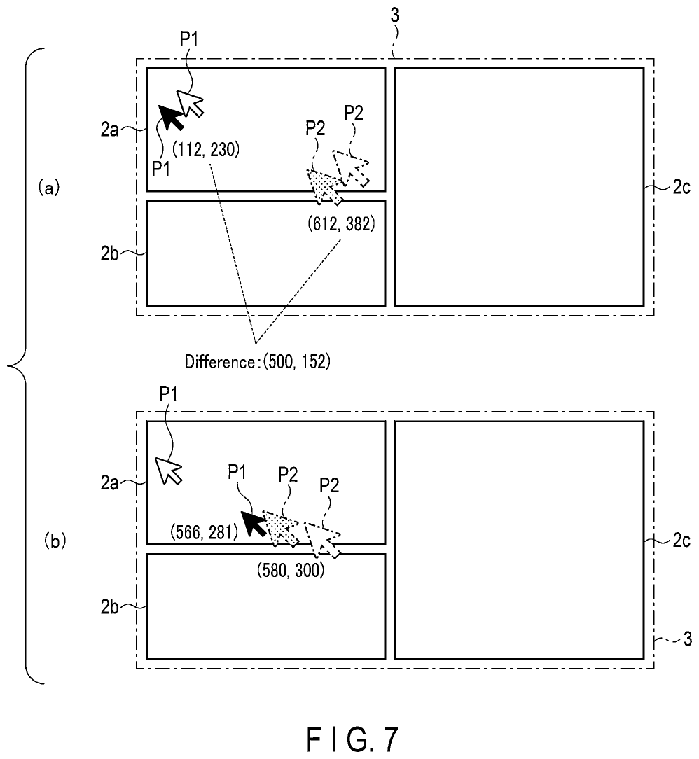

The operation of a medical information processing system including the medical information display control apparatus having the above arrangement will be described next with reference to the flowchart of FIG. 3, the schematic views of FIGS. 4 and 5, the flowchart of FIG. 6, and the schematic views of FIGS. 7, 8, and 9. Although FIGS. 4, 5, 7, 8, and 9 illustrate pointer coordinates for easy understanding, the pointer coordinates are not displayed on the display 3. However, this is not exhaustive, and the pointer coordinates may be displayed on the display 3. The same applies to each embodiment described below.

Assume that the plurality of external devices 2a to 2c transmit medical information screen data to the compositor 10 while displaying medical information screens. In this state, as shown in FIG. 3, step ST1 is started.

In step ST1, the display control unit 16 of the compositor 10 controls the display 3 so as to display the second pointer on a display screen on which a plurality of medical information screen data are laid out. With this operation, for example, as shown in FIG. 4, the display 3 displays a second pointer P2 at the position indicated by second pointer coordinates (1052, 384) on the display screen on which the plurality of medical information screen data accepted from the external devices 2a to 2c are laid out. Although the second pointer coordinates are illustrated for the sake of easy understanding, they are not displayed on the display 3.

In step ST2, the switching output unit 19 of the compositor 10 determines whether a click operation signal accepted by the mouse input unit 11 is detected. If NO, the process returns to step ST1 to repeat the operations in steps ST1 and ST2. In contrast to this, if it is determined in step ST2 that a click operation signal is detected, the switching output unit 19 starts to output a pointer movement signal in response to the click operation signal. Assume that, as indicated by (a) in FIG. 5, the display 3 has displayed the second pointer P2 at the position indicated by second pointer coordinates (634, 302). At this time, in response to a click operation signal, the switching output unit 19 starts to output a pointer movement signal to the external device 2a corresponding to the second pointer coordinates (634, 302). Upon accepting the pointer movement signal, the external device 2a draws a first pointer P1 on the medical information screen based on the pointer movement signal as indicated by (b) in FIG. 5. Note that the medical information screens respectively displayed by the external devices 2a to 2c are displayed on the display 3 in real time.

In step ST3 after step ST2, the mouse input unit 11 accepts a pointer movement signal corresponding to the operation of the mouse 1 by the user as a pointer operation signal (x/y movement amounts).

In step ST4, the screen input unit 12 acquires a plurality of medical information screen data laid out as a display screen from the plurality of external devices 2.

In step ST5, the mouse pointer detection unit 13 detects the first pointer P1 drawn on one of the plurality of medical information screen data and specifies first pointer coordinates (local coordinates) indicating the position of the first pointer P1. For example, the mouse pointer detection unit 13 detects the first pointer P1 based on a change in medical information screen data along the time series and the pointer operation signal accepted in step ST3. More specifically, the mouse pointer detection unit 13 detects, as the first pointer P1, an object, of the objects moving in the x and y directions along the time series in the medical information screen data, which has high correlation with the x/y movement amounts indicated by the pointer operation signal. Thereafter, the mouse pointer detection unit 13 specifies first pointer coordinates (local coordinates) indicating the position of the first pointer P1.

In step ST6, the mouse pointer coordinate calculation unit 14 calculates second pointer coordinates (global coordinates) indicating the position of the second pointer P2 drawn on the display screen based on the pointer operation signal accepted in step ST3.

In step ST7, the display control unit 16 controls the display 3 to display the second pointer P2 at the position indicated by the calculated second pointer coordinates (global coordinates) on the display screen.

In step ST8, the compositor 10 outputs a pointer movement signal based on the pointer operation signal so as to cause the layout management unit 15, the difference calculation unit 17, the feedback calculation unit 18, and the switching output unit 19 to almost match the first pointer coordinates with the second pointer coordinates. In this case, step ST8 includes steps ST8-1 to ST8-4, as shown in FIG. 6.

In step ST8-1, the layout management unit 15 identifies the medical information screen including the second pointer P2 from the second pointer coordinates (global coordinates) calculated in step ST6 and the held layout information. In addition, the layout management unit 15 converts the calculated second pointer coordinates (global coordinates) into local coordinates on the medical information screen including the second pointer P2. Note that step ST8-1 may be executed between steps ST6 and ST7.

In step ST8-2, the difference calculation unit 17 calculates the difference between the first pointer coordinates (local coordinates) specified in step ST5 and the second pointer coordinates (local coordinates) obtained in step ST8-1. For example, in the case of the display screen indicated by (a) in FIG. 7, the difference calculation unit 17 calculates a difference (500, 152) between first pointer coordinates (112, 230) indicating the position of the first pointer P1 after the movement to the lower left side and second pointer coordinates (612, 382) indicating the position of the second pointer P2 after the movement to the lower left side. Referring to FIG. 7, the first pointer P1 and the second pointer P2 before movement are expressed by the outlined arrows, and the first pointer P1 and the second pointer P2 after movement are expressed by the hatched arrows. Similar arrows are used in each drawing described below to represent the pointers P1 and P2 before and after movement.

In step ST8-3, the feedback calculation unit 18 calculates a feedback amount so as to almost match the first pointer coordinates with the second pointer coordinates based on the difference. For example, the feedback calculation unit 18 intermittently calculates feedback amounts. Note that the feedback calculation unit 18 may start intermittent calculation of feedback amounts in response to the reception of a difference by a mouse operation. Alternatively, the feedback calculation unit 18 may always execute intermittent calculation of feedback amounts. In this case, the feedback calculation unit 18 starts intermittent calculation of feedback amounts in response to the reception of a difference. In this case, the feedback calculation unit 18 holds data about the current calculation and uses the held data for subsequent new calculation after a time interval. That is, after an arbitrary time since the current calculation, the feedback calculation unit 18 uses data about the previous calculation for this calculation. More specifically, the feedback calculation unit 18 calculates a feedback amount from the previous difference and the ratio between the previous input mouse movement amount and the output mouse movement amount so as to bring the current different close to zero. The time intervals for the calculation of feedback amounts may be constant or occasional. If the time intervals are occasional, the time intervals may be changed in accordance with the difference such that, for example, the time intervals for calculation are shortened as the difference increases and vice versa. A feedback amount is a movement amount ratio for bringing the current difference close to zero.

If, for example, a difference D (xd, yd), first pointer coordinates (x1, y1), and second pointer coordinates (x2, y2) are set in each calculation at predetermined time intervals, the difference D is obtained as follows.

$$D = ( x 2 , y 2 ) - ( x 1 , y 1 ) = ( x 2 - x 1 , y 2 - y 1 ) = ( xd , yd )$$

In addition, let (m1, m2) be a variable m proportional to the previous difference D. The variable m is a variable for bringing the current difference close to zero. If the difference D is zero, the variable m is zero.

In addition, let (x_i, y_i) be the previous input mouse movement amount and (x_o, y_o) be the previous output mouse movement amount. Letting Rn=(x_r, y_r) be the ratio between the previous input mouse movement amount and the previous output mouse movement amount, the ratio Rn is obtained as follows.

$$Rn = ( x\_r , y\_r )$$

$$x\_r = ( x\_i + m 1 ) / x\_o ( where x\_i , x\_o > 0 )$$

$$y\_r = ( y\_i + m 2 ) / y\_o ( where y\_i , y\_o > 0 )$$

The following provides a supplementary explanation about the variable m. Assume that there is no variable m1. In this case, if the previous input mouse movement amount x_i in the x direction is 1 and the previous output mouse movement amount x_o is 0.5, a ratio Rn* is obtained as follows. Note that the same applies to the y direction.

$${Rn}^{*} = x\_i / x\_o = 1 / 0.5 = 2$$

That is, if the first pointer P1 has previously moved by only half of the previous movement amount of the mouse 1, the ratio Rn* is doubled. In the absence of the variable m1, however, the ratio Rn* in the subsequent calculations will only make the ratio between the previous input mouse movement amount x_i and the previous output mouse movement amount x_o be 1:1 but cannot reduce the current difference D. Therefore, in order to reduce the current difference D, the calculation of the ratio Rn includes the variable m proportional to the previous difference D.

Note that if the mouse 1 is in motion, an adjusted pointer movement signal is obtained by multiplying the ratio Rn by the current input mouse movement amount (x_i, y_i). The adjusted pointer movement signal corresponds to a current output mouse movement amount (x_r·x_i, y_r·y_i). Assume that the mouse 1 is in the resting state. In this case, if the previous input mouse movement amount is (0, 0) and the previous output mouse movement amount (0, 0), the adjusted pointer movement amount may be set to a movement amount (m3, m4) based on the variable m. That is, if the mouse 1 is in the resting state, the current difference D may be brought close to zero by using the pointer movement signal adjusted to the predetermined movement amount (m3, m4). The pointer movement signal adjusted to the predetermined movement amount (m3, m4) corresponds to the current output mouse movement amount (m3, m4).

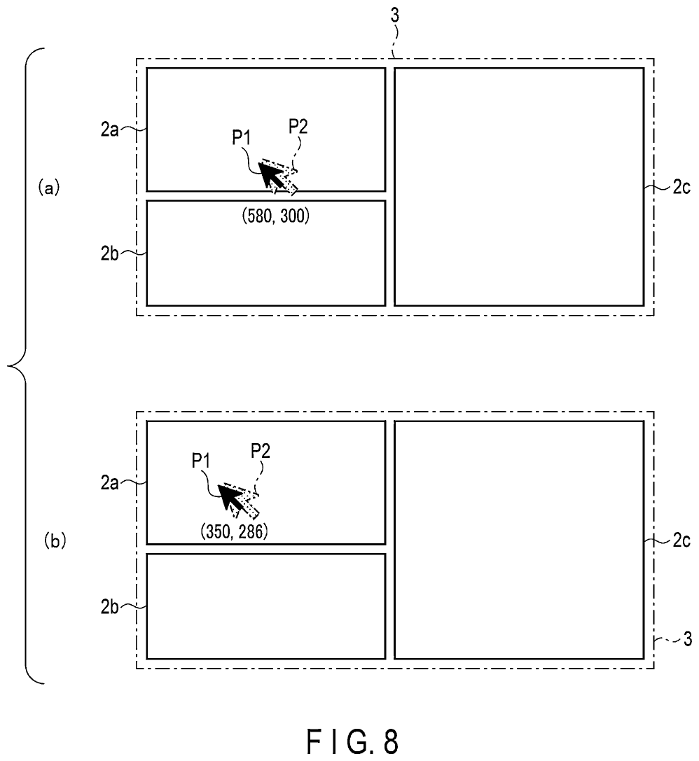

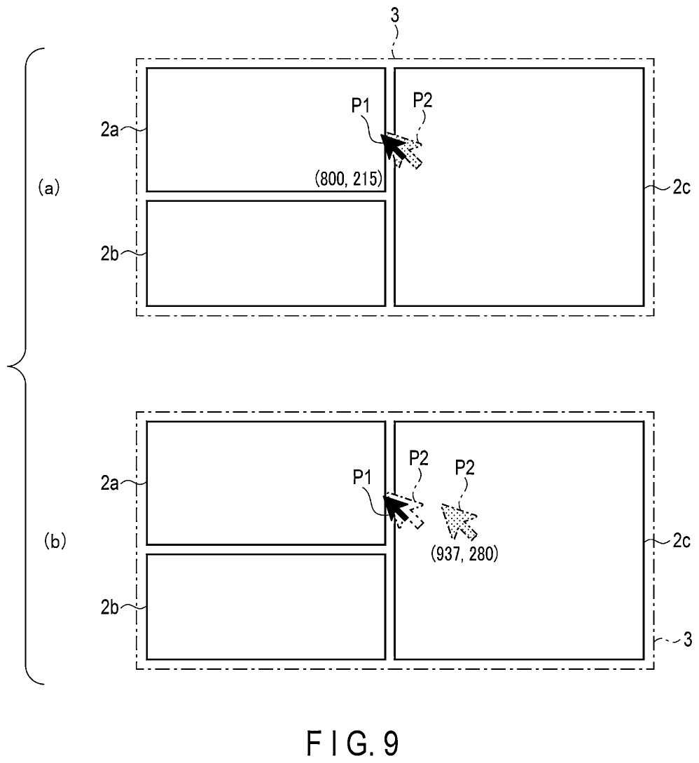

In step ST8-4, the switching output unit 19 adjusts the pointer movement signal transmitted from the layout management unit 15 based on the feedback amount and outputs the adjusted pointer movement signal to the external device 2 that displays a medical information screen including the first pointer P1. For example, the switching output unit 19 adjusts the pointer movement signal so as to almost match the first pointer coordinates with the second pointer coordinates and outputs the adjusted pointer movement signal to the external device 2. As indicated by (a) and (b) in FIG. 7, this updates the first pointer coordinates from (112, 230) to (566, 281) and updates the second pointer coordinates from (612, 382) to (580, 300), thereby gradually bringing the first pointer coordinates close to the second pointer coordinates. Along with this operation, the display 3 displays a situation in which the first pointer and the second pointer gradually come close to each other as the first pointer P1 greatly moves to lower right and the second pointer P2 moves a little to upper left. Thereafter, as indicated by (a) in FIG. 8, the display 3 displays a situation in which the first pointer coordinates (580, 300) match the second pointer coordinates (580, 300), and the first pointer P1 overlaps the second pointer P2. Note that FIG. 7 shows an example of a display screen in a case where a pointer movement signal is output upon being adjusted to match the first pointer coordinates with the second pointer coordinates by bringing them close to each other. FIG. 8 and FIG. 9 (to be described later) each show an example of a display screen in a case where a pointer movement signal is output upon being adjusted to maintain a state in which the first pointer coordinates almost match the second pointer coordinates. In addition, the first pointer P1 and the second pointer P2 have similar shapes with different sizes, and, for example, the small first pointer P1 overlaps the large second pointer P2, and hence one pointer is not hidden by the other pointer. Note that the magnitude relationship between the two pointers may be reversed such that the small second pointer P2 overlaps the large first pointer P1. In either case, when the coordinates of the two pointers almost match, step ST8-4 is terminated.

Subsequently, the first pointer P1 and the second pointer P2 are displayed such that they integrally move to the position indicated by the second pointer coordinates (350, 286) updated in accordance with the operation of the mouse 1, as indicated by (b) in FIG. 8.

Assume that, as indicated by (a) in FIG. 9, the updated second pointer coordinates (800, 215) have reached a layout boundary. In this case, the layout management unit 15 controls the switching output unit 19 based on layout information so as to stop outputting a pointer movement signal to the external device 2a corresponding to medical information screen data having undergone a transition from a state including the second pointer coordinates to a state including no second pointer coordinates. The external device 2a stops receiving a pointer movement signal and stops moving the first pointer P1 on the medical information screen.

Subsequently, as indicated by, for example, (b) in FIG. 9, the display control unit 16 controls the display 3 so as to display the second pointer P2 at the position indicated by the updated second pointer coordinates (937, 280). The display control unit 16 repeatedly executes, for example, steps ST3 to ST8 until the end of the operation by an ending operation by the user (step ST9).

As described above, according to the first embodiment, the mouse input unit 11 accepts a pointer operation signal corresponding to the operation of the mouse 1 by the user. The screen input unit 12 acquires a plurality of medical information screen data laid out as a display screen from the plurality of external device 2. The mouse pointer detection unit 13 detects the first pointer drawn on one of the plurality of medical information screen data and specifies the first pointer coordinates indicating the position of the first pointer. The mouse pointer coordinate calculation unit 14 calculates the second pointer coordinates indicating the position of the second pointer drawn on the display screen. The difference calculation unit 17, the feedback calculation unit 18, and the switching output unit 19 output a pointer movement signal based on a pointer operation signal so as to almost match the first pointer coordinates with the second pointer coordinates. As described above, the arrangement that matches the first pointer coordinates indicating the first pointer drawn on one of the plurality of medical information screen data with the second pointer coordinates indicating the second pointer drawn on the display screen can improve the operability with respect to the pointer moving across a plurality of screens.

For supplementary explanation, according to the prior art, it is assumed that the user has moved the mouse pointer from a given medical information screen to another medical information screen and has performed the (jump-in) operation of transferring the control of the mouse pointer to an external device corresponding to the medical information screen at the destination. At this time, according to the prior art, the coordinates of the mouse pointer on the display screen (drawn by the compositor) before jump-in do not match the coordinates of the mouse pointer (drawn by the OS of the external device) after the jump-in.

Likewise, according to the first embodiment, the first pointer coordinates immediately after jump-in do not match the second pointer coordinates. However, according to the first embodiment, feedback control makes it possible to instantly and automatically move the first pointer P1 on a medical information screen to the position of the second pointer P2 on a display screen.

In addition, according to the prior art, the moving speed of the mouse pointer before jump-in is different from the moving speed of the mouse pointer after the jump-in.

In contrast to this, according to the first embodiment, since the pointer movement amount to the external device 2 is calculated with reference to the second pointer coordinates on the display screen, the moving speed of the pointer with respect to a mouse operation does not change before and after jump-in. Accordingly, the first embodiment can improve the operability with respect to the pointer moving across a plurality of screens.

According to the prior art, in returning (jump-out) the control of a mouse pointer to the compositor again, an operation other than a normal mouse operation is required. In the case of the prior art, the compositor cannot detect the coordinates of the mouse pointer on a medical information screen after jump-in and hence cannot determine to perform jump-out when, for example, the mouse has reached an end of the medical information screen. In contrast to this, according to the first embodiment, since the first pointer coordinates on a medical information screen are detected, it can be known that the first pointer has moved to an end of the medical information screen. In addition, according to the first embodiment, the arrangement configured to almost match the first pointer coordinates with the second pointer coordinates can perform jump-out in response to the movement of the first pointer coordinates outside the medical information screen.

In addition, according to the first embodiment, the screen input unit 12 individually acquires a plurality of medical information screen data from the plurality of external devices 2. The switching output unit 19 outputs a pointer movement signal to one of the plurality of external devices 2. The layout management unit 15 controls the switching output unit 19 to stop outputting a pointer movement signal to the external device 2 corresponding to medical information screen data having undergone a transition from a state including the second pointer coordinates to a state including no second pointer coordinates based on layout information indicating the layout of a plurality of medical information screen data. Accordingly, in addition to having the above effects, this embodiment can stop updating and displaying the first pointer P1 with respect to the external device 2 corresponding to medical information screen data having undergone a transition from a state including second pointer coordinates to a state including no second pointer coordinates.

According to the first embodiment, the mouse pointer detection unit 13 can detect the first pointer P1 based on a change in medical information screen data along the time series and a pointer operation signal. In addition to the above effects, it is possible to detect, as the first pointer P1, for example, an object, of the objects moving along the time series in medical information screen data, which has high correlation with the pointer operation signal. Accordingly, according to the first embodiment, since the first pointer P1 can be detected without using the shape data of the first pointer P1, the first pointer P1 with an arbitrary shape can be detected.

According to the first embodiment, first pointer coordinates are local coordinates indicating a position on medical information screen data. Second pointer coordinates are global coordinates indicating a position on a display screen. The layout management unit 15 converts second pointer coordinates as global coordinates into local coordinates by associating the local coordinates of each of a plurality of medical information screen data with the global coordinates on the display screen based on layout information. The difference calculation unit 17, the feedback calculation unit 18, and the switching output unit 19 output a pointer movement signal so as to almost match the first pointer coordinates with the second pointer coordinates as the converted local coordinates. This makes it possible to obtain the above effects even if global coordinates are different from local coordinates.

According to the first embodiment, the mouse input unit 11 accepts a click operation signal corresponding to a click operation by the user. The switching output unit 19 starts to output a pointer movement signal in response to the click operation signal. In addition to the above effects, this makes it possible to prevent an operation from being started at a timing not intended by the user.

According to the first embodiment, the difference calculation unit 17, the feedback calculation unit 18, and the switching output unit 19 adjust a pointer movement signal so as to almost match the first pointer coordinates with the second pointer coordinates and output the adjusted pointer movement signal. In addition to the above effects, this makes it possible to adjust and output a pointer movement signal.

According to the first embodiment, the first pointer and the second pointer have similar shapes with different sizes. Accordingly, in addition to the above effects, after the first pointer coordinates match the second pointer coordinates, one pointer is not completely hidden by the other pointer, and hence the user can visually recognize both the pointers.

Note that the first embodiment described above may be modified as follows.

In the first embodiment, all the external devices 2 each display one medical information screen. However, this is not exhaustive, and at least one external device 2 may display a plurality of medical information screens. According to this modification, if the layout of a plurality of medical information screens on the external device 2 matches the layout in the display screen of the display 3, it is possible to regard a plurality of screens on one external device 2 as one screen. This makes it possible to implement this modification similarly to the first embodiment.

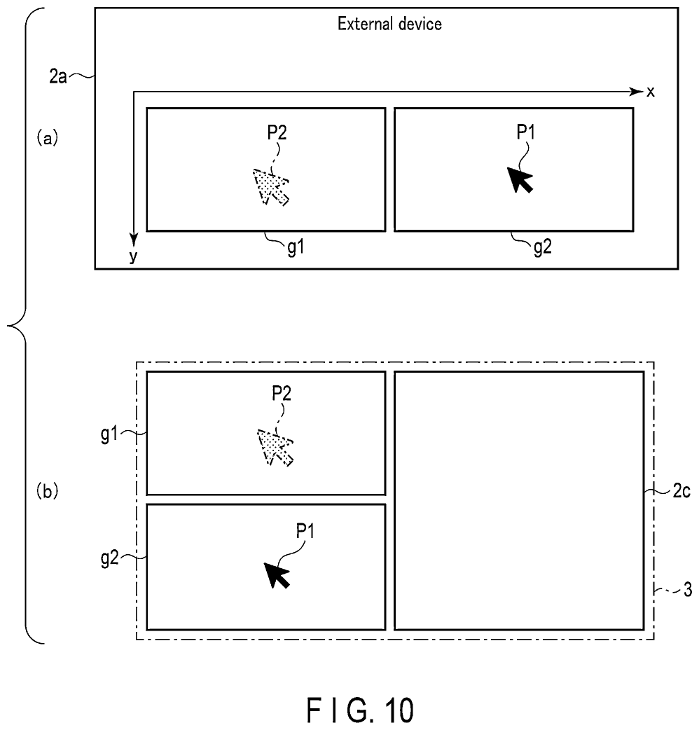

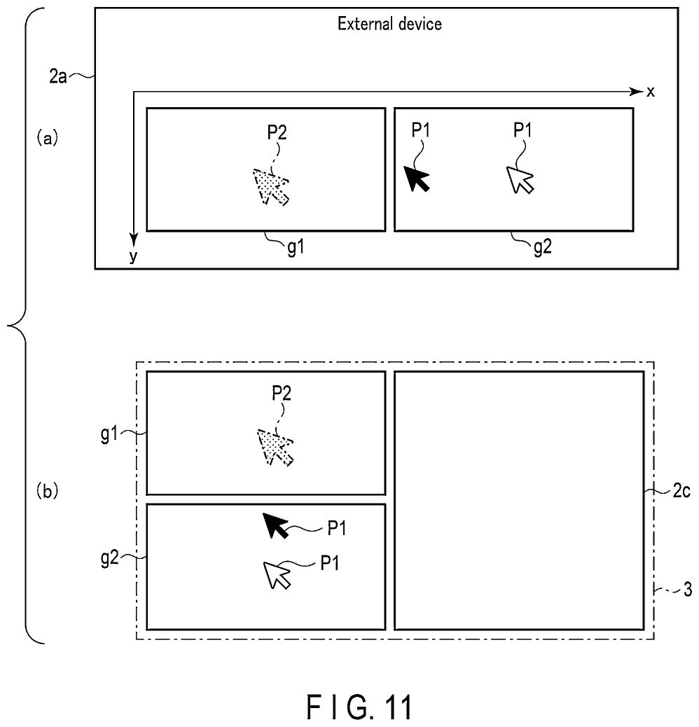

The following is a description of a case where the layout of a plurality of medical information screens on the external device 2 is different from the layout in the display screen of the display 3. Assume that as indicated by (a) in FIG. 10, one external device 2a lays out and displays a plurality of medical information screens g1 and g2 in the x direction (left/right direction). Assume also that as indicated by (b) in FIG. 10, the display 3 lays out and displays the plurality of medical information screens g1 and g2 on the external device 2a in the y direction (up/down direction). In this case, the layout information held by the layout management unit 15 indicates a situation in which the first layout of the plurality of medical information screens g1 and g2 on at least one external device 2a of the plurality of external devices 2 is different from the layout of the plurality of medical information screens g1 and g2 in the display screen on the display 3. The switching output unit 19 outputs a pointer movement signal to the external device 2a, of the plurality of external devices 2, which displays the medical information screen g2 on which the first pointer P1 is detected. If the medical information screen g2 on which the first pointer P1 is detected is the second medical information screen of one external device 2a, the compositor 10 calculates the movement amount of the pointer based on the first layout. For example, when bringing the first pointer P1 close to the second pointer P2 along the y direction on the display screen of the display 3, the compositor 10 calculates the movement amount of the pointer so as to move the first pointer P1 along the x direction on the medical information screen g2 of the external device 2a. In FIG. 11, (a) indicates a state in which the first pointer P1 is moved in the x direction on the medical information screen g2 of the external device 2a, and (b) indicates a state in which the first pointer P1 is moved in the y direction on the medical information screen g2 in the display screen of the display 3. Note that after the first pointer P1 enters the medical information screen g1 including the second pointer, the first pointer P1 may be moved between the display screen of the display 3 and the medical information screen g1 of the external device 2a along the same direction.

First of all, in specifying the first pointer coordinates (local coordinates) indicating the position of the detected first pointer P1, the compositor 10 specifies the first pointer coordinates as local coordinates on the medical information screen g2 of the external device 2a based on the layout information on the device. Likewise, in converting the second pointer coordinates (global coordinates) into the second pointer coordinates (local coordinates), the compositor 10 obtains the second pointer coordinates (local coordinates) as local coordinates on the medical information screen g1 of the external device 2a based on the first layout. With this operation, the compositor 10 calculates the movement amount of the pointer so as to reduce the difference between the first pointer coordinates and the second pointer coordinates to zero in the local coordinate systems of the medical information screens g1 and g2 of the external device 2a. Accordingly, as indicated by (a) in FIG. 11, the external device 2a displays a state in which the first pointer P1 approaches the second pointer P2 along the x direction.

On the other hand, the display control unit 16 draws the first pointer P1 on the display screen so as to bring the first pointer P1 close to the second pointer P2 based on the difference between the first pointer coordinates and the second pointer coordinates as the converted local coordinates and the second layout. More specifically, for example, the display control unit 16 stops updating the display screen based on the medical information screen data acquired from the external device 2 and displays the display screen in the resting state on the display 3. With this operation, the first pointer P1 in motion along the x direction on the medical information screen g2 of the external device 2a is not displayed on the display screen of the display 3. Thereafter, the display control unit 16 draws the first pointer P1 to bring it close to the second pointer P2 along the y direction on the display screen in the resting state based on the second layout so as to reduce the absolute value of the above difference to zero. Accordingly, as indicated by (b) in FIG. 11, the display 3 displays a state in which the first pointer P1 approaches the second pointer P2 along the y direction.

According to this modification, therefore, even if the layout of a plurality of medical information screens on one external device 2 is different from the layout in the display screen of the display 3, effects similar to those of the first embodiment can be obtained.

According to the first embodiment, after the first pointer coordinates almost match the second pointer coordinates, the first pointer P1 and the second pointer P2 are integrally moved. However, this is not exhaustive. For example, the display control unit 16 causes the display 3 to display a display screen on which the first pointer P1 and the second pointer P2 are drawn. In this case, the display control unit 16 stops drawing the second pointer when the first pointer coordinates almost match the second pointer coordinates. This makes it possible to erase the second pointer P2 and move only the remaining first pointer P1 after the first pointer coordinates almost match the second pointer coordinates.

In the first embodiment, there is no specific mention on the size of each medical information screen in a display screen and the moving speed of a pointer in each medical information screen. However, this is not exhaustive. For example, the display control unit 16 may control the moving speed of the second pointer P2 in accordance with the size of a medical information screen on which the second pointer P2 is drawn. More specifically, if a first medical information screen is larger than a second medical information screen, the display control unit 16 may adjust the moving speed of the second pointer P2 in the second medical information screen to a low speed lower than the moving speed of the second pointer P2 in the first medical information screen after the second pointer P2 in the first medical information screen moves into the second medical information screen. In this case, the low speed may be a predetermined low speed or a low speed calculated based on the ratio of the size of the second medical information screen to the size of the first medical information screen. In either case, according to this modification, when the second pointer P2 is moved in a small medical information screen, it is possible to relatively easily adjust the position of the second pointer P2.

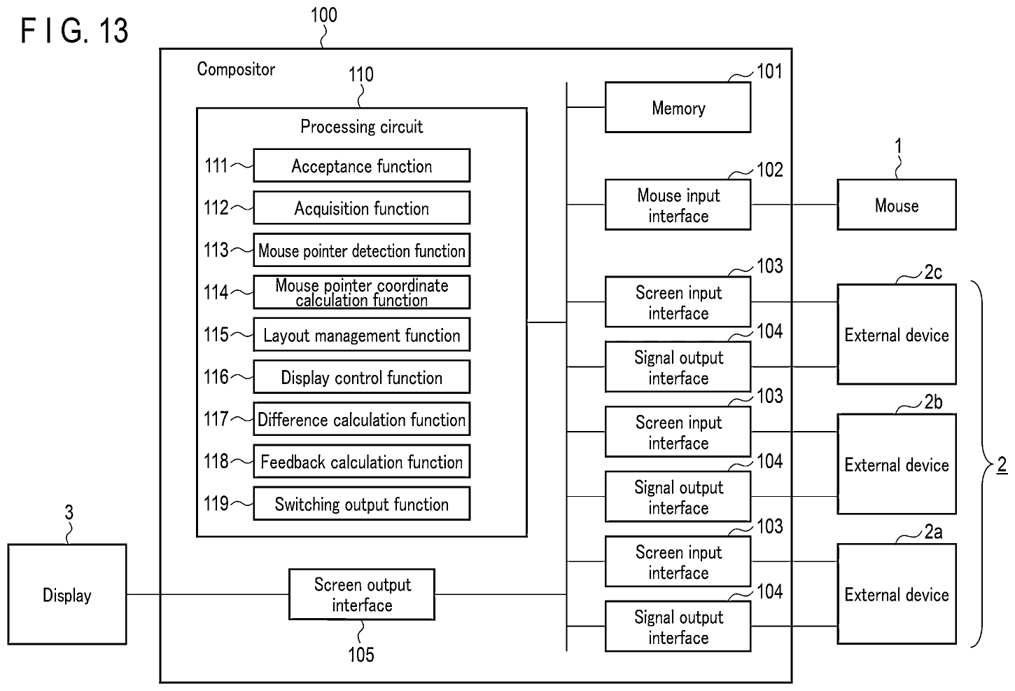

In addition, the first embodiment and each modification each have exemplified the case where the compositor 10 is implemented by the hardware arrangement. However, this is not exhaustive. For example, as shown in FIG. 13, a compositor 100 may be implemented by an arrangement in which hardware resources and software function in cooperation with each other. That is, the compositor 100 has functions similar to those of the compositor 10 described in the first embodiment and each modification.

In this case, the compositor 100 includes, as hardware resources, a memory 101, a mouse input interface 102, a screen input interface 103, a signal output interface 104, a screen output interface 105, and processing circuitry 110. The compositor 100 also includes the functions implemented by executing programs in the memory 101. These functions include an acceptance function 111, an acquisition function 112, a mouse pointer detection function 113, a mouse pointer coordinate calculation function 114, a layout management function 115, a display control function 116, a difference calculation function 117, a feedback calculation function 118, and a switching output function 119.

The memory 101 is constituted by memories recording electrical information, such as a ROM (Read Only Memory), a RAM (Random Access Memory), an HDD (Hardware Disk Drive), an image memory and peripheral circuits such as a memory controller accompanying the memories and a memory interface. The memory 101 stores, for example, various programs such as medical information control programs for the compositor (medical information display control apparatus) and various data such as layout information, data under processing, and processed data. Note that the layout information is the same as that described above.

The mouse input interface 102 is connected to the mouse 1 for inputting various commands, instructions, information, selections, and settings from the operator (user) to the compositor body and the processing circuitry 110. The mouse input interface 102 is an interface circuit that receives an electrical signal corresponding to the operation of the mouse 1 by the user and outputs the electrical signal to the processing circuitry 110.

A screen input interface 103 is connected to the plurality of external devices 2 and the processing circuitry 110. The screen input interface 103 is an interface circuit that individually receives a plurality of medical information screen data from the plurality of external devices 2 and outputs each medical information screen data to the processing circuitry 110.

A signal output interface 104 is connected to one of the plurality of external devices 2 and the processing circuitry 110. The signal output interface 104 is an interface circuit that outputs the pointer movement signal received from the processing circuitry 110 to the connected external device 2.

The screen output interface 105 is an interface circuit that outputs the display screen data received from the processing circuitry 110 to the display 3. The display screen data may have the second pointer P2 drawn or not drawn therein.

The processing circuitry 110 reads out programs stored in the memory 101 based on an instruction input by the user via the mouse input interface 102 and controls the compositor 100 in accordance with the programs. For example, the processing circuitry 110 is a processor that implements the respective functions of the compositor 100 in accordance with the programs read out from the memory 101. The processing circuitry 110 executes a medical information control method in accordance with the programs read out from the memory 101. For example, the processing circuitry 110 executes a medical information control method including accepting a pointer operation signal corresponding to an operation by the user, acquiring a plurality of medical information screen data laid out as a display screen, detecting the first pointer drawn in one of a plurality of medical information screen data and specifying the first pointer coordinates indicating the position of the first pointer, calculating the second pointer coordinates indicating the position of the second pointer drawn on a display screen based on the pointer operation signal, and outputting a pointer movement signal based on the pointer operation signal so as to almost match the first pointer coordinates with the second pointer coordinates. This program is installed from a network or a non-transitory computer-readable storage medium into the compositor 100. The processor of the compositor 100 then executes the program to implement each function of the compositor 100. Note that each function may be implemented by a plurality of processors in a distributed manner as needed. Alternatively, each function or part of each function may be implemented by other apparatuses as needed.

The acceptance function 111, the acquisition function 112, the mouse pointer detection function 113, the mouse pointer coordinate calculation function 114, the layout management function 115, the display control function 116, the difference calculation function 117, the feedback calculation function 118, and the switching output function 119, each referred to above, will be described sequentially. The task assigned to each function (to be described below) is arbitrary and can be changed as needed. This is because even if the process assigned to a given function is assigned to another function, the fact remains that the processing circuitry 110 executes the process.

The acceptance function 111 accepts a pointer operation signal corresponding to the operation of the mouse 1 by the user via the mouse input interface 102. The acceptance function 111 and the mouse input interface 102 correspond to the mouse input unit 11 described above.

The acquisition function 112 acquires a plurality of medical information screen data laid out as a display screen from the plurality of external devices 2 via the screen input interface 103. The acquisition function 112 and the screen input interface 103 correspond to the screen input unit 12 described above.

The mouse pointer detection function 113 detects the first pointer drawn in one of a plurality of medical information screen data and specifies the first pointer coordinates indicating the position of the first pointer. The mouse pointer detection function 113 corresponds to the mouse pointer detection unit 13 described above.

The mouse pointer coordinate calculation function 114 calculates the second pointer coordinates indicating the position of the second pointer drawn on the display screen based on a pointer operation signal. The mouse pointer coordinate calculation function 114 corresponds to the mouse pointer coordinate calculation unit 14 described above.

The layout management function 115 identifies a medical information screen including the second pointer from the second pointer coordinates (global coordinates) calculated by the mouse pointer coordinate calculation function 114 and layout information in the memory 101. In addition, the layout management function 115 converts the calculated second pointer coordinates (global coordinates) into local coordinates on a medical information screen including the second pointer. In addition, the layout management function 115 transmits a pointer movement signal corresponding to a pointer operation signal to the switching output function 119. The layout management function 115 and the memory 101 correspond to the layout management unit 15 described above.

The display control function 116 causes the display 3 to display a display screen via the screen output interface 105 based on display screen data including a plurality of medical information screen data laid out in accordance with layout information and the second pointer drawn in accordance with the second pointer coordinates (global coordinates). The display control function 116 and the screen output interface 105 correspond to the display control unit 16 described above.

The difference calculation function 117 calculates the difference between the first pointer coordinates (local coordinates) specified by the mouse pointer detection function 113 and the second pointer coordinates (local coordinates) converted by the layout management function 115. The difference calculation function 117 corresponds to the difference calculation unit 17 described above.

The feedback calculation function 118 calculates a feedback amount so as to almost match the first pointer coordinates with the second pointer coordinates based on the difference calculated by the difference calculation function 117. The feedback calculation function 118 corresponds to the feedback calculation unit 18 described above.

The switching output function 119 adjusts the pointer movement information transmitted by the layout management function 115 based on the feedback amount calculated by the feedback calculation function 118. The switching output function 119 outputs a pointer movement signal corresponding to the adjusted pointer movement information to one of the external devices 2 via the signal output interface 104. The switching output function 119 corresponds to the switching output unit 19 described above.

Therefore, according to the modification shown in FIG. 13, even if the compositor 100 is implemented by an arrangement in which hardware resources and software function in cooperation with each other, it is possible to obtain effects similar to those of the first embodiment and each modification.

### Second Embodiment

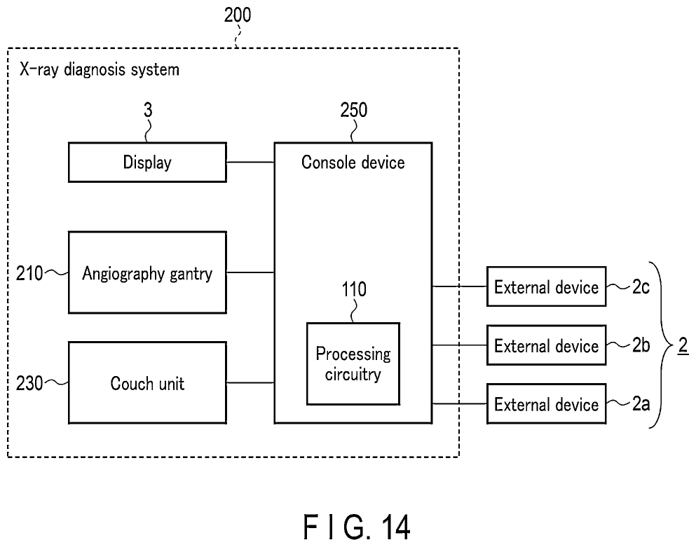

An X-ray diagnosis system according to the second embodiment will be described next with reference to FIG. 14. Note that the same reference numerals in the following description denote substantially the same constituent elements, and a detailed description of them will be omitted. Different constituent elements will be mainly described below.

The second embodiment is configured to be implemented by an X-ray diagnosis system 200 as shown in FIG. 14 unlike the first embodiment implemented by the compositors 10 and 100.

Accordingly, the X-ray diagnosis system 200 includes a display 3, an angiography gantry 210, a couch unit 230, and a console device 250. The console device 250 includes the processing circuitry 110 described above and is connected to a plurality of external devices 2.

In this case, the external device 2 and the display 3 are the same as those in the first embodiment, and the processing circuitry 110 is the same as that in the modification of the first embodiment.

The angiography gantry 210 includes a C-arm having an X-ray tube at one end and an X-ray detector at the other end, a holding unit that holds the C-arm, and a support arm that holds the holding unit at the distal end. The support arm has an almost arcuate shape and a proximal end attached to a moving mechanism with respect to a rail provided on the ceiling. The angiography gantry 210 is controlled by the console device 250 to irradiate an object placed on the couch top of the couch unit 230 with X-rays from the X-ray tube. The X-ray detector detects X-rays transmitted through the object and outputs the detection result to the console device 250.

The couch unit 230 has a couch top on which an object is placed. The couch top is held so as to be movable along the long-axis direction or the short-axis direction of the couch top and is held so as to be pivotal about the axis of the couch top in the long-axis direction.

The console device 250 controls the angiography gantry 210 and the couch unit 230 and generates and saves an X-ray image of the object based on the detection result transmitted from the angiography gantry 210. The console device 250 described above includes general components such as a memory, an input interface including a mouse, an output interface, a display, an image processing circuit, and a system control circuit. In addition to these components, the console device 250 includes the processing circuitry 110 described above. As described above, the processing circuitry 110 implements the respective functions by executing programs in the memory. The respective functions include an acceptance function 111, an acquisition function 112, a mouse pointer detection function 113, a mouse pointer coordinate calculation function 114, a layout management function 115, a display control function 116, a difference calculation function 117, a feedback calculation function 118, and a switching output function 119, each described above. The details of each function are the same as those described above.

Other components are the same as those of the first embodiment.

According to the above arrangement, in the X-ray diagnosis system 200, the processing circuitry 110 in the console device 250 accepts a pointer operation signal corresponding to an operation by the user. The processing circuitry 110 acquires a plurality of medical information screen data laid out as a display screen from the external devices 2. The processing circuitry 110 detects the first pointer P1 drawn in one of a plurality of medical information screen data and specifies the first pointer coordinates indicating the position of the first pointer P1. The processing circuitry 110 calculates the second pointer coordinates indicating the position of the second pointer P2 drawn on the display screen based on the pointer operation signal. The processing circuitry 110 outputs a pointer movement signal based on the pointer operation signal to one of the external devices 2 which draws the first pointer P1 so as to almost match the first pointer coordinates with the second pointer coordinates.

As described above, according to the second embodiment, in the X-ray diagnosis system 200, the processing circuitry 110 in the console device 250 operates similarly to the compositors 10 and 100 described above, and hence it is possible to obtain operational effects similar to those of the first embodiment.

In addition, according to the second embodiment, it is possible to improve the operability with respect to, for example, a pointer moving across a plurality of screens including X-ray images in the X-ray diagnosis system 200.

Note that in the second embodiment, the console device 250 includes the processing circuitry 110 of the compositor 100. However, this is not exhaustive. For example, the console device 250 may include the compositor 10 instead of the processing circuitry 110 of the compositor 100. Such modification can also obtain effects similar to those of the second embodiment.

### Third Embodiment

The third embodiment will be described next. The third embodiment will be described below by exemplifying the case in which the embodiment is applied to the medical information display control apparatus according to the first embodiment. However, this is not exhaustive, and the third embodiment may be applied to the X-ray diagnosis system according to the second embodiment.

The third embodiment is configured to operate in response to the arrival of the second pointer at a portion near a boundary in a medical information screen instead of a click operation signal.

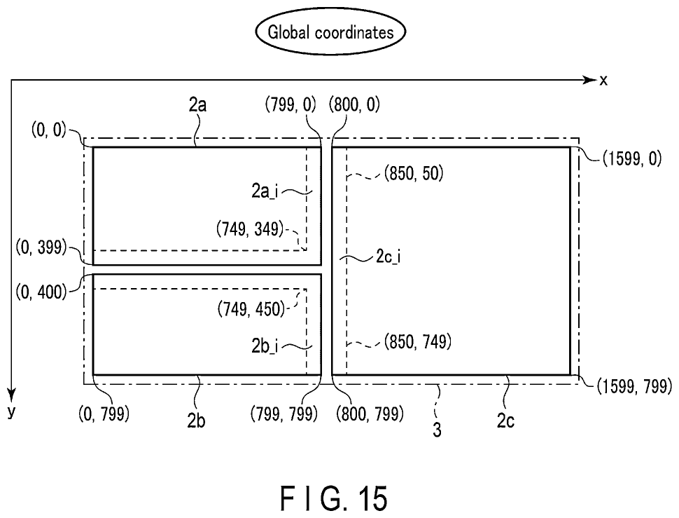

In this case, the portion near the boundary is a region near adjacent sides of the four sides of each of a plurality of adjacent medical information screens, as an example shown in FIG. 15. For example, the medical information screen displayed by an external device 2a includes a boundary neighborhood 2a_i at two sides adjacent to the medical information screens displayed by the external devices 2b and 2c. Likewise, the medical information screen displayed by the external device 2b includes a boundary neighborhood 2b_i at two sides adjacent to the medical information screens displayed by the external devices 2a and 2c. Likewise, the medical information screen displayed by the external device 2c includes a boundary neighborhood 2c_i at two sides adjacent to the medical information screens displayed by the external devices 2a and 2b.

As exemplified by FIG. 15, specific regions of boundary neighborhoods 2a_i, 2b_i, and 2c_i are set in layout information using global coordinates. In setting using global coordinates, the coordinate values of global coordinates may be input to the layout management unit 15 in advance. In addition, in setting using global coordinates, a predetermined pixel range may be input to the layout management unit 15 in advance, and the layout management unit 15 may set the coordinate values obtained by converting the pixel range into global coordinates.

Accordingly, a switching output unit 19 starts to output a pointer movement signal based on layout information indicating the layout of a plurality of medical information screen data in response to the arrival of the second pointer coordinates included in one of the plurality of medical information screen data at a boundary neighborhood of the medical information screen data. For example, the switching output unit 19 starts to output a pointer movement signal to the external device 2a in response to the arrival of the second pointer coordinates in the medical information screen data of the external device 2c at the boundary neighborhood 2a_i in the medical information screen data of the external device 2a upon movement.

Other components are the same as those of the first embodiment.

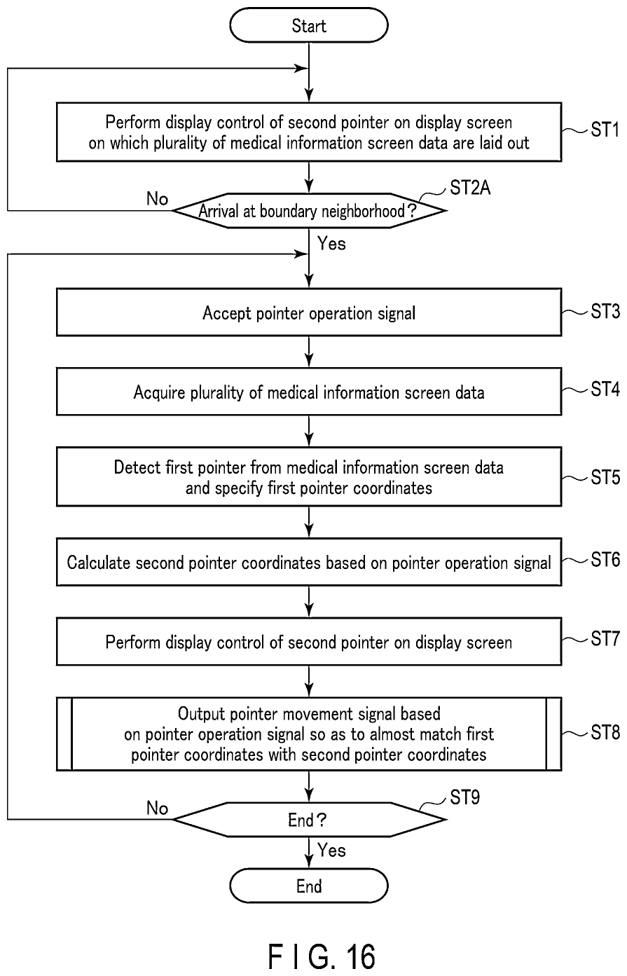

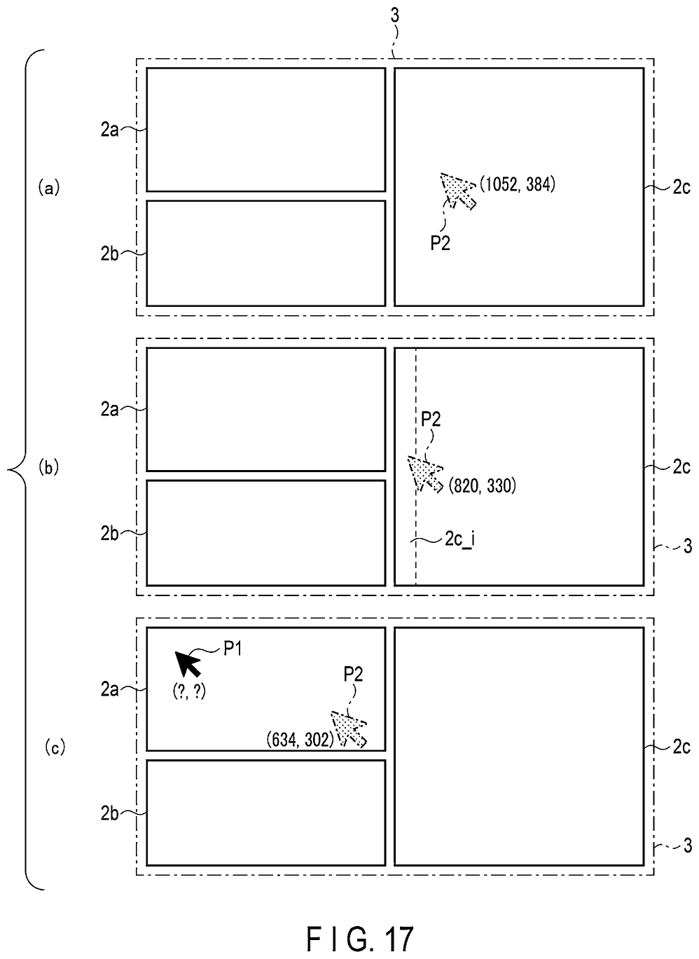

The operation of the medical information processing system including the medical information display control apparatus having the above arrangement will be described next with reference to the flow chart of FIG. 16 and the schematic view of FIG. 17.

First of all, as shown in FIG. 16 and (a) in FIG. 17, step ST1 is executed as described above.

In step ST1, a display control unit 16 of a compositor 10 controls a display 3 so as to display a second pointer on a display screen on which a plurality of medical information screen data are laid out. This causes the display 3 to display a second pointer P2 at the position indicated by second pointer coordinates (1052, 384) on a display screen on which a plurality of medical information screen data received from the plurality of external devices 2a to 2c are laid out.

In step ST2A, the switching output unit 19 of the compositor 10 determines, based on layout information, whether the second pointer coordinates have reached a boundary neighborhood of medical information screen data. If NO in step ST2A, the process returns to step ST1 to repeat the operations in steps ST1 and ST2. If YES in step ST2A, the switching output unit 19 starts to output a pointer movement signal in response to the arrival of the second pointer coordinates at the boundary neighborhood. Assume that as indicated by (b) in FIG. 17, the display 3 displays that the second pointer coordinates (820, 330) of the second pointer P2 have reached the boundary neighborhood 2c_i. At this time, the switching output unit 19 starts to output a pointer movement signal to the external device 2a corresponding to the destination of the second pointer coordinates (820, 330) in response to the arrival of the second pointer coordinates at the boundary neighborhood 2c_i. Upon receiving the pointer movement signal, the external device 2a draws the first pointer P1 on the medical information screen based on the pointer movement signal, as indicated by (c) in FIG. 17. Note that the medical information screen displayed by each of the external devices 2a to 2c is displayed on the display 3 in real time.

After step ST2A, as described above, processing in and after step ST3 is executed.

As described above, according to the third embodiment, the switching output unit 19 starts to output a pointer movement signal based on layout information indicating the layout of a plurality of medical information screen data in response to the arrival of the second pointer coordinates included in one of the plurality of medical information screen data at the boundary neighborhood 2c_i of the medical information screen data. In addition to the effects described above, this makes it possible to start to output a pointer movement signal to the external device 2a corresponding to the destination of the second pointer P2 without a jump-in operation such as a click operation by the user.

According to at least one embodiment described above, it is possible to improve the operability with respect to a pointer moving across a plurality of screens.

The term “processor” used in the above description refers to, for example, a circuit such as a CPU (Central Processing Unit, a GPU (Graphics Processing Unit), an ASIC (Application Specific Integrated Circuit), a programmable logic device (such as an SPLD (Simple Programmable Logic Device), a CPLD (Complex Programmable Logic Device), an FPGA (Field Programmable Gate Array)). The processor implements functions by reading out and executing programs stored in the memory. The programs may be directly incorporated in the circuit of the processor instead of being stored in the memory. In this case, the processor implements functions by reading out and executing programs incorporated in the circuit. Note that each processor according to the embodiments is not limited to a case in which each processor is configured as a single circuit, and a plurality of independent circuits may be combined into one processor to implement its function. In addition, a plurality of constituent elements in FIG. 1, 13, or 14 may be integrated into one processor to implement its function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information display control apparatus comprising processing circuitry configured to
accept a pointer operation signal corresponding to an operation by a user,
acquire a plurality of medical information screen data laid out as a display screen,
detect a first pointer drawn in one of the plurality of medical information screen data and specify first pointer coordinates indicating a position of the first pointer,
calculate second pointer coordinates indicating a position of a second pointer drawn on the display screen based on the pointer operation signal, and
output a pointer movement signal based on the pointer operation signal so as to substantially match the first pointer coordinates with the second pointer coordinates.

2. The apparatus according to claim 1, wherein the processing circuitry is further configured to
individually acquire the plurality of medical information screen data from a plurality of medical devices,
output the pointer movement signal to one of the plurality of medical devices, and
stop outputting the pointer movement signal to a medical device corresponding to the medical information screen data having undergone a transition from a state including the second pointer coordinates to a state not including the second pointer coordinates based on layout information indicating the layout of the plurality of medical information screen data.

3. The apparatus according to claim 1, wherein the processing circuitry is configured to detect the first pointer based on a change in the medical information screen data along time series and the pointer operation signal.

4. The apparatus according to claim 1, wherein the processing circuitry is further configured to
cause a display to display the display screen on which the first pointer and the second pointer are drawn, and
end drawing the second pointer if the first pointer coordinates substantially match the second pointer coordinates.

5. The apparatus according to claim 1, wherein the first pointer coordinates are local coordinates indicating a position in the medical information screen data,
the second pointer coordinates are global coordinates indicating a position on the display screen, and
the processing circuitry is further configured to
convert the second pointer coordinates as the global coordinates into the local coordinates by associating local coordinates of each of the plurality of medical information screen data with global coordinates on the display screen based on layout information indicating the layout of the plurality of medical information screen data, and
output the pointer movement signal so as to substantially match the first pointer coordinates with the second pointer coordinates as the converted local coordinates.

6. The apparatus according to claim 5, wherein the processing circuitry is configured to individually acquire the plurality of medical information screen data from a plurality of medical devices,
the layout information indicates a situation in which a first layout of a plurality of medical information screens on at least one medical device of the plurality of medical devices is different from a second layout of a plurality of medical information screens in a display screen on the display, and
the processing circuitry is configured to cause a display to display the display screen on which the first pointer and the second pointer are drawn and draw the first pointer and the second pointer on the display screen so as to bring the first pointer close to the second point based on a difference between the first pointer coordinates and the second pointer coordinates of the converted local coordinates and the second layout.

7. The apparatus according to claim 1, wherein the processing circuitry is configured to
accept a click operation signal corresponding to a click operation by the user, and
start to output the pointer movement signal in response to the click operation signal.

8. The apparatus according to claim 1, wherein the processing circuitry is configured to start to output the pointer movement signal in response to arrival of the second pointer coordinates included in one of the plurality of medical information screen data at a boundary neighborhood of the medical information screen data based on layout information indicating the layout of the plurality of medical information screen data.

9. The apparatus according to claim 1, wherein the processing circuitry is configured to adjust the pointer movement signal so as to substantially match the first pointer coordinates with the second pointer coordinates and output the adjusted pointer movement signal.

10. The apparatus according to claim 1, wherein the first pointer and the second pointer have similar shapes with different sizes.

11. The apparatus according to claim 1, wherein the processing circuitry is configured to
cause a display to display the display screen on which the first pointer and the second pointer are drawn, and
if a first medical information screen is larger than a second medical information screen, adjust a moving speed of the second pointer in the second medical information screen to a speed lower than a moving speed of the second pointer in the first medical information screen after the second pointer in the first medical information screen moves into the second medical information screen.

12. A medical information control method comprising:
accepting a pointer operation signal corresponding to an operation by a user,
acquiring a plurality of medical information screen data laid out as a display screen,
detecting a first pointer drawn in one of the plurality of medical information screen data and specifying first pointer coordinates indicating a position of the first pointer,
calculating second pointer coordinates indicating a position of a second pointer drawn on the display screen based on the pointer operation signal, and
outputting a pointer movement signal based on the pointer operation signal so as to substantially match the first pointer coordinates with the second pointer coordinates.

13. The method according to claim 12, wherein the acquiring includes individually acquiring the plurality of medical information screen data from a plurality of medical devices,
the outputting includes outputting the pointer movement signal to one of the plurality of medical devices, and
the method further comprises stopping outputting the pointer movement signal to a medical device corresponding to the medical information screen data having undergone a transition from a state including the second pointer coordinates to a state not including the second pointer coordinates based on layout information indicating the layout of the plurality of medical information screen data.

14. The method according to claim 12, wherein the specifying includes detecting the first pointer based on a change in the medical information screen data along time series and the pointer operation signal.

15. The method according to claim 12, further comprising causing a display to display the display screen on which the first pointer and the second pointer are drawn,
wherein the causing the display to display includes ending drawing the second pointer if the first pointer coordinates substantially match the second pointer coordinates.

16. The method according to claim 12, wherein the first pointer coordinates are local coordinates indicating a position in the medical information screen data,
the second pointer coordinates are global coordinates indicating a position on the display screen,
the method further comprises converting the second pointer coordinates as the global coordinates into the local coordinates by associating local coordinates of each of the plurality of medical information screen data with global coordinates on the display screen based on layout information indicating the layout of the plurality of medical information screen data, and
the outputting includes outputting the pointer movement signal so as to substantially match the first pointer coordinates with the second pointer coordinates as the converted local coordinates.

17. The method according to claim 16, wherein the acquiring includes individually acquiring the plurality of medical information screen data from a plurality of medical devices,
the layout information indicates a situation in which a first layout of a plurality of medical information screens on at least one medical device of the plurality of medical devices is different from a second layout of a plurality of medical information screens in a display screen on the display, and
the method further comprises causing a display to display the display screen on which the first pointer and the second pointer are drawn and drawing the first pointer and the second pointer on the display screen so as to bring the first pointer close to the second point based on a difference between the first pointer coordinates and the second pointer coordinates of the converted local coordinates and the second layout.

18. The method according to claim 12, wherein the accepting includes accepting a click operation signal corresponding to a click operation by the user, and
the outputting includes starting to output the pointer movement signal in response to the click operation signal.

19. The method according to claim 12, wherein the outputting includes starting to output the pointer movement signal in response to arrival of the second pointer coordinates included in one of the plurality of medical information screen data at a boundary neighborhood of the medical information screen data based on layout information indicating the layout of the plurality of medical information screen data.

20. An X-ray diagnosis system comprising processing circuitry configured to
accept a pointer operation signal corresponding to an operation by a user,
acquire a plurality of medical information screen data laid out as a display screen,
detect a first pointer drawn in one of the plurality of medical information screen data and specify first pointer coordinates indicating a position of the first pointer,
calculate second pointer coordinates indicating a position of a second pointer drawn on the display screen based on the pointer operation signal, and
output a pointer movement signal based on the pointer operation signal so as to substantially match the first pointer coordinates with the second pointer coordinates.
